(19) United States
(12) Patent Application Publication
(10) Pub. No.: US 2020/0326354 A1
(43) Pub. Date: Oct. 15, 2020

(54) LAMELLAR BODY PURIFICATION FOR DIAGNOSIS AND TREATMENT OF A DISEASE OR DISORDER

(71) Applicant: SIME DIAGNOSTICS LTD., London (GB)

(72) Inventors: Peter SCHOUSBOE, Odense (DK); Henrik VERDER, Bagsværd (DK)

(73) Assignee: SIME DIAGNOSTICS LTD., London (GB)

(21) Appl. No.: 16/754,019

(22) PCT Filed: Oct. 5, 2018
(86) PCT No.: PCT/EP2018/077078

§ 371 Date: Apr. 6, 2020

(30) Foreign Application Priority Data
Oct. 6, 2017 (EP) 17195174.2

(51) Int. Cl. G01N 33/92; G01N 1/28; G01N 1/38; G01N 33/68; G01N 21/3577; H01J 49/26

(52) U.S. Cl. CPC G01N 33/92; G01N 1/286; G01N 1/38; G01N 33/6848; G01N 21/3577; A61B 2010/0061; G01N 2001/2866; G01N 2800/52; G01N 2560/00; G01N 2405/04; H01J 49/26

(57) ABSTRACT

The present invention relates to methods for diagnosing diseases and disorders by measuring the amounts of one or more compounds in lamellar bodies isolated from a sample derived from a subject suspected of suffering from a disorder. Also disclosed are methods for monitoring efficacy of a treatment, methods for monitoring disease progression in a subject, as well as computer-implemented methods for diagnosis and systems for performing said methods.

### TECHNICAL FIELD

The present invention relates to methods for diagnosing diseases and disorders by measuring the amounts of one or more compounds in lamellar bodies isolated from a sample derived from a subject suspected of suffering from a disorder. Also disclosed are methods for monitoring efficacy of a treatment, methods for monitoring disease progression in a subject, as well as computer-implemented methods for diagnosis and systems for performing said methods.

### BACKGROUND

Lamellar bodies are also termed lamellar granules, membrane-coating granules (MCGs), keratinosomes or Odland bodies. They are lipid storage and secretory organelles found in type II alveolar cells in the lungs, and in keratinocytes in the skin. They are oblong structures, appearing about 300-400 nm in length and 100-150 nm in width in transmission electron microscopy images. Lamellar bodies fuse with the cell membrane and release pulmonary surfactant into the extracellular space. They are surrounded by a membrane and contain multilamellar lipid membranes. They may also contain apolipoproteins and lytic enzymes and have an acidic pH. Under normal physiological conditions, their main function is the supply of extracellular domains with specialised lipid components related to a specialised function. The lamellar bodies of the lung epithelium are the storage form of lung surfactant. They provide a monomolecular lipid film of dipalmitoyl phosphatidylcholine on the surface of alveoli to lower surface tension necessary for optimal gas exchange. They also provide a hydrophobic protective lining against environmental influences. Lamellar bodies are also found in other cell types of the respiratory system, for example the mucosa of the nose and the bronchia.

The gastrointestinal tract, the tongue papillae, the oral epithelium and mucosal cells of the stomach also contain lamellar bodies. Phosphatidylcholine is the major phospholipid of lamellar bodies in mucosa cells of the stomach, providing a hydrophobic protective lipid film against the tissue-damaging activities of gastric juice.

The hydrophobic water-protective barrier of the skin also originates from lamellar bodies secreted by epithelial cells, and consists mainly of neutral lipids. Lamellar bodies also occur in mesodermal cell layers of sliding surfaces to provide joint lubrication, as well as in the peritoneum, the pericardium and the pleural mesothelium.

Lamellar bodies have also been found to accumulate in several pathological conditions, such as atherosclerosis, Niemann-Pick disease. The fact that lysosomal lamellar bodies are absent in the normal intima of the aortic wall, but appear in cells in fatty streaks might indicate that their formation is relevant to the pathogenetic mechanisms which become involved in the development of atherosclerosis.

Methods for diagnosing diseases and disorders are best suited for point-of-care units if they can be used to establish a reliable diagnosis with a short time-to-result, preferably using small sample volumes.

The present inventors have found that analysing the contents of lamellar bodies isolated from various body samples can be used to establish such diagnosis.

### DESCRIPTION OF DRAWINGS

FIG. 1 Left panel: frozen and thawed gastric aspirate samples show mucus-like, flocculent material, mainly composed of phospholipids and proteins. Right panel: fresh gastric aspirate samples do not show flocculent material.

FIG. 2 Analysis of crude GAS and purified LB fractions analysed by MS show high correlations between L/S ratio and RDS. The horizontal line shows an appropriate cut-off value (3.0) for determining whether a newborn suffers from RDS. Sensitivity of the method is 91%, and specificity is 81%.

FIG. 3 Upper panel: electron microscopy of precipitated lamellar bodies from GAS at birth. Lower panel: lamellar bodies from GAS in large electron microscopy magnification.

### SUMMARY

The invention is as defined in the claims. The inventors have found that lamellar bodies can be concentrated from a body sample by a combination of separation methods, preferably a forced separation, such as centrifugation. The result is that the supernatant can be discarded from the sample and the lamellar bodies are concentrated in a pellet. The lamellar bodies are then resuspended for analysis, e.g. analysis of their contents.

Herein is provided method for analysing lamellar bodies, said method comprising the steps of:
i) centrifuging a sample to obtain a pellet comprising lamellar bodies, and a supernatant;
ii) discarding the supernatant and resuspending the pellet, thereby obtaining a sample for analysis;
iii) analysing the sample.

Also provided herein is a method of treatment of a disease or a disorder in a subject, comprising:
i) performing a diagnosis method described herein, thereby determining whether said individual suffers or is likely to suffer from a disease or a disorder; and
ii) treating said subject.

Also provided is a method of monitoring progression of a disease or a disorder in a subject, comprising:
i) preparing samples comprising lamellar bodies for analysis as described herein, where the samples are representative of different time points;
ii) analysing the samples;
iii) comparing the analysis results over time to monitor the progression of the disease or disorder.

Also provided herein is a method of monitoring treatment efficacy, comprising:
i) Administering a treatment to a subject suffering from a disease;
ii) preparing samples comprising lamellar bodies for analysis as described herein, where the samples are representative of different time points of the treatment;
iii) analysing the samples;
iv) comparing the analysis results over time to monitor treatment efficacy.

Also provided herein is a computer implemented method for diagnosing a disease or disorder based on data acquired from a sample obtained from a subject, the method comprising the steps of:
i) acquiring data for the sample,
ii) correlating said data with a control value, wherein a predetermined difference is indicative of the subject suffering from said disease or disorder.

Also provided herein is a computer program product having a computer readable medium, said computer program product being suitable for diagnosing a disease or disorder in a subject based on data acquired from a sample obtained from said subject, said computer program product comprising means for carrying out all the steps of the diagnosis methods described herein.

### DETAILED DESCRIPTION OF THE INVENTION

### Definitions

### Analysis Means

The term ‘analysis means’ as used herein refers to an instrument capable of detecting the physical property of a molecule or group of molecules. In one embodiment the analysis means is an FTIR spectrometer capable of performing measurements in very small sample volumes such as down to 1 μL.

### Mid-IR

The term Mid-IR or Mid wavelength infrared, also called intermediate infrared (IIR) and mid-red FTIR spectroscopy as used herein refers to light having a wavelength of between about 3 to about 50 μm.

### Diagnosis Methods

Early diagnosis may be essential for successfully treating, preventing or slowing down progression of a disease or disorder. This can be achieved by measuring the amount of one or more compound of interest in a sample comprising lamellar bodies.

The present inventors have found that measuring the amount of a compound of interest in the lamellar bodies contained in a sample obtained from a subject can be used to reproducibly and reliably diagnose a disease or disorder. The methods require but minute sample volumes and may be performed with a short time-to-result, whereby the methods are particularly well suited for point of care units, without time-consuming laboratory preparations of the sample.

For example, the present methods are believed to be useful for measuring the amounts of compounds such as lecithin or saturated lecithin and sphingomyelin in lamellar bodies which are comprised within the sample. This can be used e.g. to determine whether a newborn suffers from Respiratory Distress Syndrome. This is described in co-pending application entitled “Fetal lung maturity test” assigned to the same applicant and having the same filing date as the present application.

In a clinical setting, the physician utilising the present methods may, based on the result of the diagnostic method, apply the method of exclusion to determine if the subject wherefrom the sample has been obtained is indeed suffering from a disease or disorder. If the result of the method indicates an amount of the one or more compounds of interest which is significantly different from a control ratio, the subject is diagnosed with the disorder or disease.

### Samples

In a first step, a sample is provided, wherein the sample is obtained from a subject, in particular a subject suffering or suspected of suffering from a disease or disorder. The present methods may be performed on any body sample comprising lamellar bodies. Such samples may be e.g. an epithelium sample, a gastric aspirate sample, an amniotic fluid sample, a sample of the joints e.g. a synovial fluid sample, a gastrointestinal sample, a blood sample or an oropharyngeal secretion.

In some cases, care has to be taken to avoid contamination of the sample. For example, if the sample is obtained from amniotic fluid, care should be taken to prevent contamination of the amniotic fluid. In one embodiment, the sample is obtained from a subject, such as a human being e.g. a female, such as a pregnant female.

The chances of collecting non-contaminated or essentially non.contaminated amniotic fluid are good in connection with caesarean sectioning. Thus in one embodiment the subject is a female human being, undergoing, or immediately about to undergo, caesarean sectioning. In a further embodiment the body fluid sample is amniotic fluid collected from the female human being, during or immediately subsequent to the caesarean sectioning.

As mentioned above, the present method allows for handling very small sample volumes. In one embodiment, the sample has a volume between 10 and 1000 μL, such as between 10 and 750 μL, such as between 20 and 500 μL, such as between 30 and 250 μL, such as between 40 and 125 μL, such as between 50 and 100 μL, such as between 60 and 90 μL, such as between 70 and 80 μL, such as 50 μL, 75 μL or 100 μL. In some embodiments, the sample has a volume less than 1000 μL, such as less than 900 μL, such as less than 800 μL, such as less than 700 μL, such as less than 600 μL, such as less than 500 μL, such as less than 400 μL, such as less than 300 μL, such as less than 200 μL, such as less than 100 μL, such as less than 90 μL, such as less than 80 μL, such as less than 70 μL, such as less than 60 μL, such as less than 50 μL. In specific embodiments, the volume of the sample is 50 μL, 75 μL or 100 μL.

Preferably, the sample is untreated prior to performing the present methods. Care should be taken however to try and obtain a sample which is as homogeneous as possible. Step ii) of the methods preferably comprises a step of homogenising the sample, as described below.

In particular, the sample is preferably not frozen prior to performing the present methods. The samples may if needed be stored at low temperatures for up to several weeks prior to analysis by the present methods. For example, storage has been found not to affect the phospholipid content as shown in the examples. Storage may be for 1 hour or more, such as 2 hours or more, such as 3 hours or more, such as 6 hours or more, such as 12 hours or more, such as 24 hours or more, such as 2 days or more, such as 3 days or more, such as 1 week or more, such as 2 weeks or more, such as 1 month. Storage is preferably at a temperature between 1 and 10° C., such as 2° C., 3° C., 4° C., 5° C., 6° C., 7° C., 8° C., 9° C. or 10° C., preferably at 4° C. or 5° C.

### Homogenisation of the Sample

In step ii), the sample may be diluted and/or homogenised in a first solution. Homogenisation can be achieved as is known to the skilled person. For example, the sample may be placed on a vortex, thereby strongly stirring the sample. In some embodiments, the sample may be diluted prior to and/or after homogenisation.

The sample is diluted in a first volume of a first solution. Preferably, the first solution is a hypotonic solution. The first solution may thus be water, such as deionized water, or tap water. In another embodiment, the first solution is deionized water. In a third embodiment, the first solution is plain water, such as tap water. Without being bound by theory, it is hypothesized that the first solution lyses the cells, thereby facilitating precipitation of lamellar bodies in the next steps of the method.

The volume of the first solution can vary, since the sample is precipitated in later steps of the method. The volume of the first solution should preferably be at least equal to half the volume of the sample, such as at least equal to the volume of the sample, such as at least equal to twice the volume of the sample, such as at least equal to three times the volume of the sample, such as at least equal to four times the volume of the sample, such as at least equal to five times the volume of the sample, such as at least equal to 6 times the volume of the sample, such as at least equal to 7 times the volume of the sample, such as at least equal to 8 times the volume of the sample, such as at least equal to 9 times the volume of the sample, such as at least equal to 10 times the volume of the sample, or more. Thus in one embodiment, the ratio of the volume of the sample of step i) to the volume of the first solution used in step 2 is 1:0.5. In another embodiment the ratio of the sample of step i) to the volume of the first solution used in step ii) is 1:1. In another embodiment the ratio of the sample of step i) to the volume of the first solution used in step ii) is 1:2. In another embodiment the ratio of the sample of step i) to the volume of the first solution used in step ii) is 1:3. In another embodiment the ratio of the sample of step i) to the volume of the first solution used in step ii) is 1:4. In another embodiment the ratio of the sample of step i) to the volume of the first solution used in step ii) is 1:5. In another embodiment the ratio of the sample of step i) to the volume of the first solution used in step ii) is 1:6. In another embodiment the ratio of the sample of step i) to the volume of the first solution used in step ii) is 1:7. In another embodiment the ratio of the sample of step i) to the volume of the first solution used in step ii) is 1:8. In another embodiment the ratio of the sample of step i) to the volume of the first solution used in step ii) is 1:9. In another embodiment the ratio of the sample of step i) to the volume of the first solution used in step ii) is 1:10.

After adding the volume of the first solution to the sample of step i), the sample and the first solution are mixed and homogenized, e.g. by vortexing or pipetting as is known to the skilled person, until the mixture appears homogenous to the naked eye. A homogenous sample is thereby obtained.

### Precipitation of the Lamellar Bodies

In step iii) of the method, the homogenous sample obtained in step ii) is transferred to a centrifuge, and centrifugation is performed in order to obtain a pellet comprising lamellar bodies and a supernatant. In step iv), the supernatant is discarded.

Centrifugation is performed as is known in the art, at a force and for a duration sufficient to allow the lamellar bodies to be precipitated from the sample, so that a pellet is obtained comprising lamellar bodies. Without being bound by theory, centrifugation is thought to allow removal of substantially all or almost all the cellular debris.

Centrifugation may be performed at a force between 500 and 10000 g, such as between 1000 and 9000 g, such as between 2000 and 800 g, such as between 3000 and 7000 g, such as between 3500 and 6000 g, such as between 3750 and 5000 g, such as between 3750 and 4500 g, such as at about 4000 g. Centrifugation may be performed for a duration of 1 min to 10 min, such as 2 min to 9 min, such as 3 min to 8 min, such as 4 min to 7 min, such as 5 min to 6 min, such as 4 min, 5 min or 6 min.

For example, a centrifugation step of 4 minutes at 4000 g is suitable for performing the methods of the invention. Alternatively, the centrifugation may be for 2 minutes at 5000 g or more, for example 6000 g or more, for example 7000 g or more, for example 8000 g or more, for example 9000 g or more, for example 10000 g or more. The centrifugation may be for 10 minutes at 500 g or more, for example 1000 g or more, such as 2000 g or more, for example 3000 g or more, such as 4000 g or more, for example 5000 g or more, such as 6000 g or more, for example 7000 g or more, such as 8000 g or more, for example 9000 g or more, for example 10000 g.

Following centrifugation, the sample should now present two phases: a solid phase, or pellet, located at the bottom of the tube, and which may be invisible to the naked eye; and a liquid phase, or supernatant. The pellet comprises lamellar bodies from the sample in a concentrated form. The supernatant may also comprise a portion of lamellar bodies; however, the majority of the lamellar bodies is preferably present in the pellet.

The supernatant is discarded as is known in the art. This may be done by pipetting the supernatant away, while being careful not to disturb the pellet, or it may be done by simply gently pouring the supernatant away, and optionally pipetting the remaining volume. The container in which the sample is comprised may be tipped gently and tapped gently on a piece of e.g. absorbing paper, in order to remove the small volumes of liquid which may remain on the walls of the container by gravity.

In some embodiments, the method is temperature-independent at least when performed in a temperature range between 20° C. and 40° C.

### Analysis of the Sample

Once the pellet is essentially free of supernatant, it is resuspended in a volume of a second solution, so that a sample for analysis is obtained. The second solution may be a hypotonic solution or a saline solution. The second solution may be water, such as deionized water, or tap water. In one embodiment of the method, the second solution is saline solution. In another embodiment, the second solution is deionized water. In a third embodiment, the second solution is plain water, such as tap water.

In some embodiments, it may be desirable to remove at least part or all of the second liquid after resuspension or prior to analysis. This can be done for example by evaporation of at least part or all of the second liquid. In some embodiments, the method thus comprises a step of drying the sample after resuspension and/or prior to determining the amount of the first compound and/or of sphingomyelin.

The volume of second solution to be added to the pellet depends on the analysis means used in step v), and will be described in more detail below.

The amount of one or more compounds of interest in the sample for analysis can then be determined.

The one or more compounds of interest are compounds which can help diagnose a given disease or disorder. Some disorders or diseases may be characterized by the presence or absence of one or more compounds, or an amount of said compounds which is smaller than a control value may be characteristic of a disease or disorder. Conversely, other disorders or diseases may be characterized by an amount of said compounds which is greater than a control value.

The one or more compounds of interest may be a lipid, such as a sphingolipid, a phospholipid or a fatty acid.

One possible application of the present methods concerns determining the amount of a given molecule known to be involved in inflammation response mechanisms, e.g. in synovial fluid samples isolated from joints. The present methods may thus be used to diagnose e.g. rheumatoid arthritis.

In some embodiments, the one or more compounds of interest is a single compound. In other embodiments, the one or more compounds of interest is two or more compounds, such as three or more compounds, such as four or more compounds, such as five compounds or more.

The methods may further comprise performing additional steps, e.g. calculating a ratio or a difference between two of the compounds.

For example, determining the amounts of lecithin (or saturated lecithin) and sphingomyelin can be used to determine a ratio termed the L/S ratio, which can be indicative of respiratory distress syndrome, as described in co-pending application “fetal lung maturity test” filed by the same applicant and having the same filing date as the present application. Likewise, determining the concentration of lecithin (or saturated lecithin) can be used to determine a concentration of lecithin or saturated lecithin, which can be indicative of respiratory distress syndrome, as described in co-pending application “fetal lung maturity test” filed by the same applicant and having the same filing date as the present application.

The volume of sample for analysis suitable for determining the amounts of the one or more compounds, such as of a first compound and of sphingomyelin, may vary.

In some embodiments, the sample for analysis may be transferred to a CaF₂ window. Removal of at least part of the second solution may thus aptly be performed at the time of transfer, for example if the CaF₂ window is at a high temperature allowing for evaporation, such as 80° C. or more, such as 85° C., 86° C., 87° C., 88° C., 89° C., 90° C., 91° C., 92° C., 93° C., 94° C., 95° C. or more. Part of the second solution may also have been evaporated prior to this step, and in some embodiments the method thus comprises two steps of removing at least part of the second solution prior to determining the amounts of the one or more compounds.

In some embodiments, the sample for analysis is analysed using an infrared spectrometer. In a particular embodiment, the analysis means is a Fourier transformed infrared spectrometer (FTIR). Preferably, the amounts of sphingomyelin and of the first compound are determined in the mid-wavelength infrared range.

Suitable sample volumes for spectrometer analysis, in particular FTIR analysis, may be between 10 and 300 μL, such as between 25 and 175 μL, such as between 50 and 150 μL, such as between 75 and 125 μL, such as 100 μL, 75 μL, 50 μL, or 25 μL.

The amount of a compound can be measured as a concentration or as an activity, as the person of skill is well aware of. In some embodiments of the disclosure, the amount of a compound is thus its concentration and/or activity.

For example, the concentration of lecithin or saturated lecithin in the lamellar bodies in a sample may be used to diagnose RDS by comparing the concentration measured from a sample comprising lamellar bodies to a control value. The control value in this case corresponds substantially to the concentration measured in subjects which do not suffer from RDS.

In some embodiments, the control value is 49.0 μmol/L±0.5 μmol/L, such as 49.0 μmol/L. A concentration of lecithin or saturated lecithin equal to or less than 49.0 μmol/L±0.5 μmol/L is indicative of the subject suffering from RDS. In this case, the subject may be treated as is known in the art, for example as described herein. In some embodiments, the control value is between 45.0 μmol/L±0.5 μmol/L and 53 μmol/L±0.5 μmol/L, such as between 46.0 μmol/L±0.5 μmol/L and 52 μmol/L±0.5 μmol/L, such as between 47 μmol/L±0.5 μmol/L and 51 μmol/L±0.5 μmol/L, such as between 48 μmol/L±0.5 μmol/L and 50 μmol/L±0.5 μmol/L, such as 49.0 μmol/L ±0.5 μmol/L.

In some embodiments, a ratio between the amounts of two or more compounds of interest is calculated and compared to a control ratio, where the control ratio is characteristic of healthy subjects.

For example, the L/S ratio in the lamellar bodies in a sample may be used to diagnose RDS by comparing the ratio measured from a sample comprising lamellar bodies to a control ratio. The control ratio is in this case corresponds substantially to the L/S ratio measured in subjects which do not suffer from RDS.

The control ratio for L/S is between 1.0 and 2.5±0.5. An L/S ratio smaller than the control ratio is indicative of the subject suffering from RDS. In this case, the subject may be treated as is known in the art. Accordingly, if the L/S ratio in the lamellar bodies as measured by the methods disclosed herein is less than 1.0±0.5, such as less than 1.2±0.5, such as less than 1.5±0.5, such as less than 1.7±0.5, such as less than 2.0±0.5, such as less than 2.2±0.5, such as less than 2.5±0.5, the subject is classified as having or likely to have RDS. Preferably, the control ratio is 2.0±0.5 or 2.5±0.5.

Accordingly, if the L/S ratio in the lamellar bodies as measured by the methods disclosed herein is equal to or less than 1.0±0.5, such as equal to or less than 1.2±0.5, such as equal to or less than 1.5±0.5, such as equal to or less than 1.7±0.5, such as equal to or less than 2.0±0.5, such as equal to or less than 2.2±0.5, such as equal to or less than 2.5±0.5, the subject is classified as having or likely to have RDS. Preferably, the control ratio is 2.0±0.5, 2.5±0.5 or 3.0±0.5. In other embodiments, if the L/S ratio in the lamellar bodies as measured by the methods disclosed herein is equal to or less than 2.5±0.5, such as equal to or less than 2.6±0.5, such as equal to or less than 2.7±0.5, such as equal to or less than 2.8±0.5, such as equal to or less than 2.9±0.5, such as equal to or less than 3.0±0.5, the subject is classified as having or likely to have RDS. In some embodiments, if the L/S ratio in the lamellar bodies as measured by the methods disclosed herein is less than 1.0±0.5, such as less than 1.2±0.5, such as less than 1.5±0.5, such as less than 1.7±0.5, such as less than 2.0±0.5, such as less than 2.2±0.5, such as less than 2.5±0.5, the subject is classified as having or likely to have RDS. Preferably, the control ratio is 2.0±0.5, 2.5±0.5 or 3.0±0.5. In other embodiments, if the L/S ratio in the lamellar bodies as measured by the methods disclosed herein is equal to or less than 2.5±0.5, such as equal to or less than 2.6±0.5, such as equal to or less than 2.7±0.5, such as equal to or less than 2.8±0.5, such as equal to or less than 2.9±0.5, such as equal to or less than 3.0±0.5, the subject is classified as having or likely to have RDS. In some embodiments, if the L/S ratio in the lamellar bodies as measured by the methods disclosed herein is less than 2.5±0.5, such as less than 2.6±0.5, such as less than 2.7±0.5, such as less than 2.8±0.5, such as less than 2.9±0.5, such as less than 3.0±0.5, the subject is classified as having or likely to have RDS.

The present methods preferably have a specificity of 50 or more, such as 60 or more, such as 70 or more, such as 80 or more, such as 90 or more. The present methods preferably have a sensitivity of 50 or more, such as 60 or more, such as 70 or more, such as 80 or more, such as 90 or more.

The present methods may be performed fast, and are thus well suited for point-of-care units. In some embodiments, the time-to-result of the method is between 5 and 60 minutes, such as between 10 and 50 minutes, such as between 20 and 40 minutes, such as between 25 and 35 minutes, such as about 30 minutes. In some embodiments, the time-to-result of the method is 60 minutes or less, such as 55 minutes or less, such as 50 minutes or less, such as 45 minutes or less, such as 40 minutes or less, such as 35 minutes or less, such as 30 minutes or less, such as 25 minutes or less, such as 20 minutes or less, such as 15 minute or less, such as 10 minutes or less, such as 5 minutes or less.

The time-to-result is herein defined as the time between steps i) and step vi) of the methods.

### Methods of Treatment

The present methods are useful for determining, based on a sample obtained from a subject, whether the subject suffers from a disorder or disease.

Any of the embodiments disclosed herein, i.e. any of the methods described above, may further include a step of treating a subject classified as having or likely to have a disorder or a disease. In some embodiments, the treatment is administration of a therapeutically effective amount of a therapeutic agent to the subject. The therapeutic agent may be any agent which is known or hypothesised in the art to have a therapeutic activity against said disorder or disease. The term “therapeutic agent” shall herein be construed as chemical agents or compounds having a chemical or biochemical activity which can help alleviating the symptoms of or treating a disorder or disease, as well as any other therapy in a broad sense, which may relieve or remove symptoms of the disorder or disease. The term may for example refer to e.g. change of lifestyle or psychotherapy.

In one embodiment, the method is used to determine a concentration of lecithin or saturated lecithin on a sample derived from said subject, usually newborn, to determine whether the subject suffers from RDS. In another embodiment, the method is used to determine an L/S ratio based on a sample derived from said subject, usually a newborn, to determine whether the subject suffers from RDS. The sample may be a gastric aspirate sample, an amniotic fluid sample, a blood sample or an oropharyngeal secretion sample. The concentration of lecithin or saturated lecithin can be compared to a control value, and/or the L/S ratio can be compared to a control ratio, as described in co-pending application entitled “Fetal maturity lung test” filed by the same applicant and having the same filing date as the present application, thereby indicating whether the subject suffers from Respiratory Distress Syndrome. If the subject suffers from RDS, treatment is administered—in this particular case, a therapeutically amount of surfactant is administered to the subject.

### Methods of Monitoring Disease Progression

The present methods may also be useful to monitor progression of a disorder or a disease. Herein is thus disclosed a method of monitoring progression of a disease or a disorder in a subject, comprising:
i) providing samples at different points in time from said subject, wherein said samples comprise lamellar bodies;
ii) performing steps ii) to v) as defined herein above on each of the samples, thereby determining the amount of one or more compound of interest in the sample for analysis using analysis means;
iii) comparing the amounts of the one or more compounds determined in step ii) to each other, wherein an increase or a decrease in the amount over time is indicative of a progression of the disease or disorder or of a remission of the disease or disorder.

The methods may thus be used to monitor the amounts of the one or more compounds of interest over time, thereby determining whether the amounts vary over time. In some embodiments, an increase over time is indicative of a progression of the disease or disorder. In other embodiments, a decrease over time is indicative of a progression of the disease or disorder. In other embodiments, an increase over time is indicative of a remission of the disease or disorder. In other embodiments, a decrease over time is indicative of a remission of the disease or disorder. The term remission is to be understood as the absence of disease activity for chronic disorders or diseases, or more generally as the disappearance of the disease or disorder, i.e. it may indicate that the subject no longer suffers from the disease or disorder. By contrast, progression of the disease or disorder generally indicates a worsening of the subject's condition.

In some embodiments, a stagnation of the amounts of the one or more compounds of interest indicates a stagnation of the disease or disorder, i.e. neither improvement nor worsening of the subject's condition.

In some embodiments, the amounts of the one or more compounds of interest are used to calculate a value such as a ratio or a difference. In such embodiments, changes in the ratio or difference can be indicative in a worsening or an improvement of the subject's condition.

### Methods of Monitoring Treatment Efficacy

The present methods may also be useful for determining whether a treatment is efficacious in treating or relieving symptoms of a given disease or disorder. The term “treatment” should be broadly construed as described above, and also includes treatments which are not based on chemically therapeutic compounds.

Accordingly is provided herein a method of monitoring treatment efficacy, comprising:
i) administering a treatment to a subject suffering from a disease;
ii) obtaining one or more samples from said subject at two or more subsequent points in time,
iii) performing the diagnosis method described herein on each of the samples, thereby determining the amount of one or more compound of interest in the sample for analysis using analysis means;
iv) comparing the amounts of the one or more compounds determined in step ii) to each other, wherein an increase or a decrease in the amount over time can be correlated to treatment efficacy.

The methods may thus be used to monitor the amounts of the one or more compounds of interest over time, thereby determining whether the treatment has an effect on the disease or disorder. In some embodiments, an increase over time is indicative of the treatment being efficacious against the disease or disorder. In other embodiments, a decrease over time is indicative of the treatment not being efficacious against the disease or disorder. In other embodiments, an increase over time is indicative of of the treatment not being efficacious against the disease or disorder. In other embodiments, a decrease over time is indicative of the treatment being efficacious against the disease or disorder.

In some embodiments, a stagnation of the amounts of the one or more compounds of interest indicates that the treatment is efficacious against the disease or disorder, i.e. neither improvement nor worsening of the subject's condition. This may be relevant for diseases or disorders where there is no cure, but where treatments merely aim at relieving symptoms or slowing down progression of the disease or disorder.

In some embodiments, the amounts of the one or more compounds of interest are used to calculate a value such as a ratio or a difference. In such embodiments, changes in the ratio or difference can be indicative in a worsening or an improvement of the subject's condition, and can be used to indicate whether a treatment is efficacious or not.

### Computer Implemented Method and Systems for Diagnosis

In one aspect, the invention concerns a computer implemented method for diagnosing a disease or disorder based on spectral data acquired from sample obtained from a subject, the method comprising the steps of:
i) determining the activity and/or concentration of one or more compounds by acquiring spectral data for the sample,
ii) correlating said activity and/or concentration with a control value, wherein an activity and/or concentration differing from the control value is indicative of the subject suffering from said disease or disorder.

Step i) may be performed by any of the methods described herein above, particularly in the section entitled “diagnosis methods”.

As time may be an important factor to successfully treat or slow down progression of a disorder or disease, the diagnosis may advantageously be integrated in a diagnosis system that can be installed in hospital departments, such as the neonatal department, e.g. in the delivery room. Such a system can integrate spectroscopy, analysis and disease indication that may provide a diagnostic within minutes after a biological sample has been obtained. Accordingly is provided herein a system for diagnosing a disease or disorder on a system obtained from said subject, wherein the sample comprises lamellar bodies, said method comprising
a spectroscope for measuring spectral data from said sample,
processing means configured for

a) determining the activity and/or concentration of one or more compounds in said sample by analysing said spectral data,

b) correlating said activity and/or concentration with a control value, and

c) indicating whether the activity and/or concentration is different from the control value, wherein a predefined difference is indicative of the subject suffering from said disease or disorder.

Thus, the present computer implemented method may be may be integrated in a personal computer or it may be effectuated from a website, mobile phone, smartphone or other electronic device capable of executing computer code. A further embodiment of the invention therefore relates to a computer program product having a computer readable medium, said computer program product suitable for diagnosing a respiratory disease of a subject based on spectral data acquired from a sample obtained from said subject, said computer program product comprising means for carrying out all the steps of the herein disclosed method, wherein the sample is any sample as described above.

The system may be part of a health monitoring system as described in WO 2008/019695 disclosing a health monitoring service based on a central server, wherein the measurement of the samples are carried out as a local measurement and the measurement data are subsequently sent to a central server, where the data are processed and analysed, for example by expert knowledge systems, and a health profile is generated and sent back to the local system. Thus, the processing means may be fully or partly integrated in a central service remote from the local hospital department or even remote from the hospital. However, the processing means may also be fully integrated in the local system such that the system located in the hospital department includes spectrometer, spectral analysis and processing and disease indication.

### REFERENCES

1. Dorland's Medical Dictionary—“Neonatal respiratory distress syndrome”
2. Rodriguez R J, Martin R J, and Fanaroff, A A. (2002) Neonatal-perinatal medicine: Diseases of the fetus and infant; 7th ed. (2002):1001-1011. St. Louis: Mosby.

### 3. Kamper J, Wulff K, Larsen C, Lindequist S. (1993) Acta Paediatr; 82:193-197.

### 4. Polin R A, Sahni R. (2002) Semin Neonatol 7:739-789.

### 5. Verder H. (2007) Acta Pædiatr 96:482-484.

### 6. Verder H, Albertsen P, Ebbesen F, Greisen G, Robertson B, Bertelsen A, Agertoft L, Djernes B, Nathan E, Reinholdt J. (1999) Pediatrics 103:e24.

7. Sandri F, Plavka R, Ancora G, Simeoni U, Stranak Z, Martinelli S, Mosca F, Nona J, Thomson, M, Verder H, Fabbri L, Halliday H. (2010) Pediatrics 125:e140.

### 8. Bevilacqua G, Parmagiani S, Robertson B. (1996) J Perinat Med 24:1-12.

9. Verder H., “Prænatal bestemmelse af lungematuriteten og forebyggelse af idiopatisk respiratory distress syndrom. Lecithinsphingomyelin ratio i amnionvæsken” Doctoral dissertation 27 Nov. 1980 at University of Copenhagen.

### 10. Soll R F. (1999) Cochrane Database Syst Rev 4:CD001456.

### 11. Stevens T P, Blennow M, Meyers E H, Soll R. (2007) Cochrane Database Syst Rev 2007; 4: CD003063.

### 12. Verder H, Robertson B, Greisen G, Ebbesen F, Albertsen P, Lundstrøm K, Jacobsen T. (1994) N Engl J Med 331:1051-1055.

### 13. Soll R F. (2012) Neonatology 102:169-171.

### 14. Van Kaam A H, Jaegere A P, Borensztajn D, Rimensberger P C (2011) Neonatology 100:71-77.

### 15. Liu K-Z, Dembinski T C, Mantsch H H (1998) Prenatal Diagnosis 18: 1267-1275

16. Verder H, Heiring C, Clark H, Sweet D, Jessen T E, Ebbesen F, Björklund L J, Andreasson B, Bender L, Bertelsen A, Dahl M, Eschen C, Fenger-Grøn J, Hoffmann S F, Höskuldsson A, Brussgaard-Mouritsen M, Lundberg F, Postle A D, Schousboe P, Schmidt P, Stanchev H, Sørensen L (2017) Acta Paediatr. 2017 March; 106(3):430-437

### ITEMS

1. A method for analysing lamellar bodies, said method comprising the steps of:
i) providing a sample from a subject, wherein said sample comprises lamellar bodies;
ii) optionally diluting and homogenising said sample in a first volume of a first solution, thereby obtaining a homogenous sample;
iii) centrifuging the homogenous sample to obtain a pellet comprising the lamellar bodies, and a supernatant;
iv) discarding the supernatant and resuspending the pellet in a second volume of a second solution, thereby obtaining a sample for analysis;
v) determining the amount of one or more compound of interest in the sample for analysis using analysis means;
vi) comparing the amount measured in step v) with a control value, wherein an amount differing from the control value is indicative of the subject having or being likely to have a disorder or disease.
   2. The method according to item 1, wherein the sample is a sample selected from an epithelium sample, a gastric aspirate sample, a blood sample, an amniotic fluid sample, a sample of the joint, a gastrointestinal sample and an oropharyngeal secretion.
   3. The method according to any one of the preceding items, wherein the sample has a volume between 10 and 1000 μL, such as between 10 and 750 μL, such as between 20 and 500 μL, such as between 30 and 250 μL, such as between 40 and 125 μL, such as between 50 and 100 μL, such as between 60 and 90 μL, such as between 70 and 80 μL, such as 50 μL, 75 μL or 100 μL.
   4. The method according to any one of the preceding items, wherein the sample provided in step i) is homogenous.
   5. The method according to any one of the preceding items, wherein the first solution is a hypotonic solution.
   6. The method according to item 5, wherein the first solution is a hypotonic solution such as water or deionised water.
   7. The method according to any one of the preceding items, wherein the ratio of the volume of the sample of step i) to the volume of the first solution used in step ii) is between 1:1 and 1:10, such as 1:1, 1:2, 1:3, 1:4, 1:5, 1:6, 1:7, 1:8, 1:9 or 1:10, preferably 1:4 or 1:6.
   8. The method according to any one of the preceding items, wherein homogenising in step ii) is performed by pipetting repeatedly or vortexing.
   9. The method according to any one of the preceding items, wherein the centrifugation of step iii) is performed at a force between 500 and 10000 g, such as 4000 g.
   10. The method according to any one of the preceding items, wherein the centrifugation of step iii) is performed for a duration of 1 min to 10 min, such as 2 min to 9 min, such as 3 min to 8 min, such as 4 min to 7 min, such as 5 min to 6 min, such as 4 min, 5 min or 6 min.
   11. The method according to any of the preceding items, wherein the centrifugation of step iii) is performed at 4000 g for 4 min.
   12. The method according to any one of the preceding items, wherein discarding the supernatant in step iv) is performed by pipetting the supernatant or by pouring away the supernatant.
   13. The method according to any one of the preceding items, wherein resuspending the pellet in step iv) is performed by pipetting repeatedly or vortexing.
   14. The method according to any one of the preceding items, wherein the second solution is a hypotonic solution or saline solution.
   15. The method according to item 14, wherein the first solution is a hypotonic solution such as water or deionised water.
   16. The method according to item 14, wherein the first solution is saline solution.
   17. The method according to any one of the preceding items, wherein the second volume is between 10 and 200 μL, such as between 25 and 175 μL, such as between 50 and 150 μL, such as between 75 and 125 μL, such as 100 μL, 75 μL, 50 μL, or 25 μL.
   18. The method according to any one of the preceding items, wherein step iv) further comprises a step of drying the sample after resuspension, whereby the second solution is at least partially removed by evaporation.
   19. The method according to any one of the preceding items, wherein step v) further comprises a step of drying the sample prior to determining the amount of the one or more compounds.
   20. The method according to any one of the preceding items, wherein step v) further comprises a step of transferring the sample for analysis to a support structure such as a CaF₂ window, optionally wherein the support structure is at a temperature allowing for at least partial evaporation of the second solution, such as 90° C.
   21. The method according to any one of the preceding items, wherein the analysis means is selected an infrared spectrometer, such as a Fourier transformed infrared (FTIR) spectrometer.
   22. The method according to any one of the preceding items wherein the analysis means is an FTIR spectrometer.
   23. The method according to any one of the preceding items, wherein the amount of the one or more compounds are determined in the mid-wavelength infrared range.
   24. The method according to any one of the preceding items, wherein the one or more compounds is one compound, two compounds, three compounds, four compounds, five compounds or more.
   25. The method according to any one of the preceding items, wherein the amount of the one or more compounds is determined by measuring its activity and/or concentration.
   26. The method according to any one of the preceding items wherein the subject is a human being, such as a newborn, a premature newborn, an infant, a child, or an adult.
   27. The method according to any one of the preceding items, wherein the subject is suspected of suffering from a disease or a disorder.
   28. The method according to item 27, wherein the amount of the one or more compounds is indicative of said disease or disorder.
   29. The method according to any one of the preceding items, wherein the time-to-result of the method is between 5 and 60 minutes, such as between 8 and 30 minutes, such as 15 minutes.
   30. The method according to any one of the preceding items, wherein steps ii) to v) are performed in 60 minutes or less, such as 30 minutes or less, such as 15 minutes or less, such as 10 minutes or less.
   31. A method of treatment of a disease or a disorder in a subject, comprising:
i) performing the method of any one of the preceding items, thereby determining whether said individual suffers or is likely to suffer from a disease or a disorder; and
ii) treating said subject.
   32. A method of monitoring progression of a disease or a disorder in a subject, comprising:
i) providing samples at different points in time from said subject, wherein said samples comprise lamellar bodies;
ii) performing steps ii) to v) as defined in any one of items 1 to 29 on each of the samples, thereby determining the amount of one or more compound of interest in the sample for analysis using analysis means;
iii) comparing the amounts of the one or more compounds determined in step ii) to each other, wherein an increase or a decrease in the amount over time is indicative of a progression of the disease or disorder or of a remission of the disease or disorder.
   33. A method of monitoring treatment efficacy, comprising:
i) Administering a treatment to a subject suffering from a disease;
   ii) Obtaining one or more samples from said subject at two or more subsequent points in time,
iii) Performing the method according to any one of items 1 to 29 on each of the samples, thereby determining the amount of one or more compound of interest in the sample for analysis using analysis means;
   iv) comparing the amounts of the one or more compounds determined in step ii) to each other, wherein an increase or a decrease in the amount over time can be correlated to treatment efficacy.
   34. A computer implemented method for diagnosing a disease or disorder based on data acquired from a sample obtained from a subject, the method comprising the steps of:
i) determining the activity and/or concentration of one or more compounds by acquiring data for the sample,
ii) correlating said activity and/or concentration with a control value, wherein an activity and/or concentration differing from the control value is indicative of the subject suffering from said disease or disorder.
   35. The computer implemented method of item 34, further comprising the features of any of items 1 to 29.
   36. A computer program product having a computer readable medium, said computer program product suitable for diagnosing a disease or disorder in a subject based on data acquired from a sample obtained from said subject, said computer program product comprising means for carrying out all the steps of the method as defined in any of items 34 to 35.

### EXAMPLES

A diagnostic test for lung maturity for optimal treatment of respiratory distress syndrome (RDS) has previously been developed based on mid-infrared spectroscopy on gastric aspirates (GAS) [16]. The study was based on analyses of lecithin/sphingomyelin (SM) raiot (L/S) on frozen and thawed GAS. Lecithin was measured as dipalmitoylphosphatidylcholine (DPPC).

In the present study, analyses were performed on fresh GAS. The spectroscopy signal has been enhanced by concentrating the surfactant and problems with interfering proteins, salts and mucus-like, flocculent protein cloths have been avoided.

The method is based on FTIR technology to analyse the contents of precipitated lamellar bodies. Stable measurements by dry transmission require a short path length for the infrared beam passing through the sample. The method if thus focused on removing irrelevant and excess material such as proteins and salts, resulting in improving purity of the lamellar bodies to be analysed.

### Methods

GAS obtained immediately after birth were stored at 4° C. and analysed; some samples were analysed immediately, some were analysed within a few hours, others a few days, with a maximal storage of 2 weeks.

The L/S algorithm was built on 85 GAS (DPPC (55 samples) and SM (85 samples)) obtained from infants with gestational age 24-36 weeks. Sampling for FTIR and reference samples were obtained by standard methods. 200 μL GAS were diluted 4 fold with water and centrifuged at 4000 g for 4 minutes. After removal of the supernatant, the samples were resuspended in 100 μL of water and split in 2 aliquots of 50 μL. One aliquot was analysed by FTIR, and one aliquot was analysed by mass spectrocscopy (MS) for measuring contents in phosphatidylcholine (PC) and sphingomyelin. MS was performed as described in [16].

Dry transmission of samples was performed on CaF₂ windows (1 mm thick, 13 mm in diameter, Chrystran). The 50 μL samples were applied onto the CaF₂ windows dried on a hotplate (90° C.). The FTIR measurements were performed with Bruker Tensor 27, equipped with a DTGS detector (60 scans, resolution 4 cm⁻¹).

Pellets from treated samples were fixated in 4% paraformaldehyde until preparation for electron microscopy scanning.

### Results

### Viscosity of Frozen and Fresh Samples

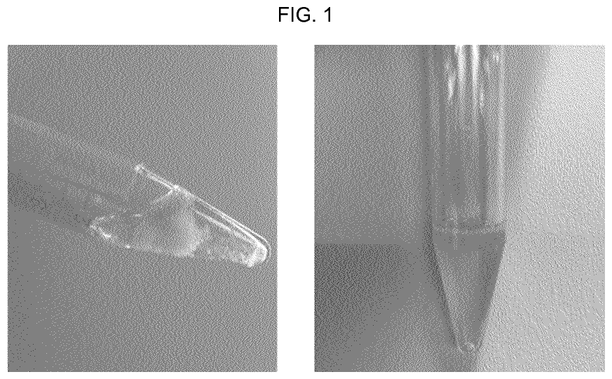

Fresh, frozen and thawed GAS were compared in 30 cases. The mucus-like, flocculent material composed of phospholipids, proteins and mucus appeared mainly to be a consequence of freezing and was observed in both the frozen and the thawed material. These clot-like structures were mostly insoluble in contrast to fresh gastric aspirates which despite a high viscosity did not display clot formation, and could be dissolved and diluted (FIG. 1).

### Mass Spectroscopy of Proteins and Phospholipids

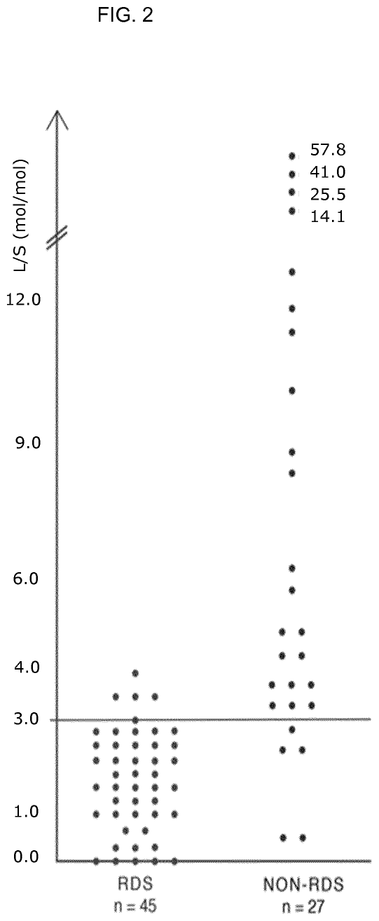

MS of proteins and phospholipids revealed that the mucus-like, flocculent material was consisting of a wide range of proteins and phospholipids. Protein content showed that mucus-like was dominant in the GAS. Further analysis also showed a high concentration of phospholipids. However, analysis of crude GAS and purified LB fractions analysed by MS show high correlations (FIG. 2).

### Stability of Phospholipids During Storage

Four fresh GAS from newborns with various gestational age were included. The PC and SM contents were measured by MS at birth and again after storage for four weeks at 4-5° C. The phospholipids were stable and unchanged during the period.

### Electron Microscopy

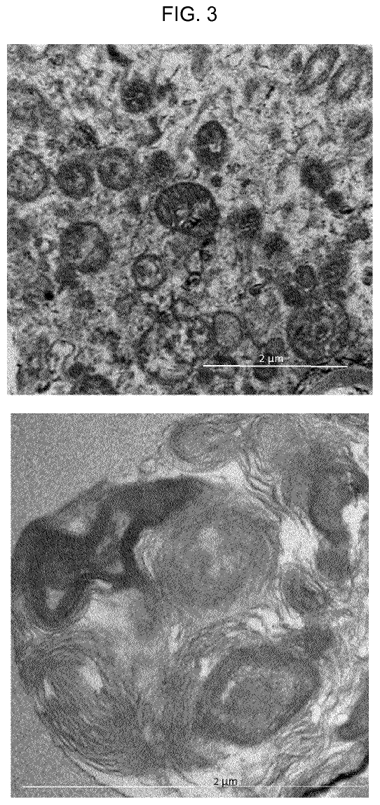

The lamellar bodies were visualized by electron microscopy (FIG. 3). Pellets obtained from GAS diluted with water showed lamellar body structures in samples from neonates at various gestational ages.

### Sensitivity and Specificity

The present method shows a sensitivity of 91% and a specificity of 79% based on 72 neonate GAS. By comparison, diagnosis on a cut-off value (control ratio) for the L/S ratio of 3.0 and DPPC contents alone has a sensitivity of 93% and a specificity of 74%.

### Blood Samples

The method was also applied to blood samples. The present method appears to reduce uncertainties as the blood cells during the hypotonic conditions burst and are removed along with the supernatant. MS of phospholipids indicated that most PC and SM originate from lamellar bodies.

### CONCLUSION

We have developed a method for use with dry transmission that reduces salt and protein contents in the samples, thus resulting in stable and reliable measurements. Dilution of the samples lowers the viscosity of the allowing lamellar bodies to be precipitated by centrifugation at low g-force, where most cellular debris, proteins and salts remain in the supernatant. These improvements leave a smaller amount of more relevant material in the form of lamellar bodies carrying the surfactant. Water is evaporated by drying the samples, for example on a hotplate. Furthermore, the method is temperature independent, at least in the range of 20 to 40° C.

Using an appropriate spectroscope, lung maturity can thus be measured and determined within the first 10 to 15 minutes of life, with high specificity and sensitivity.

1. A method for analysing lamellar bodies, said method comprising the steps of:
i) providing a sample from a subject, wherein said sample comprises lamellar bodies;
ii) optionally diluting and homogenising said sample in a first volume of a first solution, thereby obtaining a homogenous sample;
iii) centrifuging the homogenous sample to obtain a pellet comprising the lamellar bodies, and a supernatant;
iv) discarding the supernatant and resuspending the pellet in a second volume of a second solution, thereby obtaining a sample for analysis;
v) determining the amount of one or more compound of interest in the sample for analysis using analysis means;
vi) comparing the amount measured in step v) with a control value, wherein an amount differing from the control value is indicative of the subject having or being likely to have a disorder or disease.

2. The method according to claim 1, wherein the sample is a sample selected from an epithelium sample, a gastric aspirate sample, a blood sample, an amniotic fluid sample, a sample of the joint, a gastrointestinal sample and an oropharyngeal secretion.

3. The method according to any one of the preceding claims, wherein the sample has a volume between 10 and 1000 μL, such as between 10 and 750 μL, such as between 20 and 500 μL, such as between 30 and 250 μL, such as between 40 and 125 μL, such as between 50 and 100 μL, such as between 60 and 90 μL, such as between 70 and 80 μL, such as 50 μL, 75 μL or 100 μL.

4. The method according to any one of the preceding claims, wherein the first solution is a hypotonic solution such as water or deionised water, or saline solution, and/or wherein the ratio of the volume of the sample of step i) to the volume of the first solution used in step ii) is between 1:1 and 1:10, such as 1:1, 1:2, 1:3, 1:4, 1:5, 1:6, 1:7, 1:8, 1:9 or 1:10, preferably 1:4 or 1:6.

5. The method according to any one of the preceding claims, wherein the centrifugation of step iii) is performed at a force between 500 and 10000 g, such as 4000 g, for a duration of 1 min to 10 min, such as 2 min to 9 min, such as 3 min to 8 min, such as 4 min to 7 min, such as 5 min to 6 min, such as 4 min, 5 min or 6 min.

6. The method according to any one of the preceding claims, wherein the second solution is a hypotonic solution such as water or deionised water, or saline solution.

7. The method according to any one of the preceding claims, wherein the analysis means is selected an infrared spectrometer, such as a Fourier transformed infrared (FTIR) spectrometer.

8. The method according to any one of the preceding claims, wherein the one or more compounds is one compound, two compounds, three compounds, four compounds, five compounds or more, and/or wherein the amount of the one or more compounds is determined by measuring its activity and/or concentration.

9. The method according to any one of the preceding claims wherein the subject is a human being, such as a newborn, a premature newborn, an infant, a child, or an adult, optionally wherein the subject is suspected of suffering from a disease or a disorder.

10. The method according to claim 9, wherein the amount of the one or more compounds is indicative of said disease or disorder.

11. The method according to any one of the preceding claims, wherein steps ii) to v) are performed in 60 minutes or less, such as 30 minutes or less, such as 15 minutes or less, such as 10 minutes or less.

12. A method of monitoring progression of a disease or a disorder in a subject, comprising:
i) providing samples at different points in time from said subject, wherein said samples comprise lamellar bodies;
ii) performing steps ii) to v) as defined in any one of claims 1 to 11 on each of the samples, thereby determining the amount of one or more compound of interest in the sample for analysis using analysis means;
iii) comparing the amounts of the one or more compounds determined in step ii) to each other, wherein an increase or a decrease in the amount over time is indicative of a progression of the disease or disorder or of a remission of the disease or disorder.

13. A method of monitoring treatment efficacy, comprising:
i) Administering a treatment to a subject suffering from a disease;
ii) Obtaining one or more samples from said subject at two or more subsequent points in time,
iii) Performing the method according to any one of claims 1 to 11 on each of the samples, thereby determining the amount of one or more compound of interest in the sample for analysis using analysis means;
iv) comparing the amounts of the one or more compounds determined in step ii) to each other, wherein an increase or a decrease in the amount over time can be correlated to treatment efficacy.

14. A computer implemented method for diagnosing a disease or disorder based on data acquired from a sample obtained from a subject, the method comprising the steps of:
i) determining the activity and/or concentration of one or more compounds by acquiring data for the sample,
ii) correlating said activity and/or concentration with a control value, wherein an activity and/or concentration differing from the control value is indicative of the subject suffering from said disease or disorder,
optionally further comprising the features of any of claims 1 to 11.

15. A computer program product having a computer readable medium, said computer program product suitable for diagnosing a disease or disorder in a subject based on data acquired from a sample obtained from said subject, said computer program product comprising means for carrying out all the steps of the method as defined in claim 14.
